(19) United States
(12) Patent Application Publication
(10) Pub. No.: US 2008/0191696 A1
(43) Pub. Date: Aug. 14, 2008

(54) Patient Support With Integrated Spine Coil

(75) Inventor: Olaf Van Der Burgt, Eindhoven (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 11/917,009

(22) PCT Filed: Jun. 1, 2006
(86) PCT No.: PCT/IB2006/051763

§ 371 Date: Dec. 10, 2007

Related U.S. Application Data
(60) Provisional application No. 60/689,393, filed on Jun. 10, 2005.

(51) Int. Cl. G01R 33/28; A61B 5/055

(52) U.S. Cl. 324/318; 600/415

(57) ABSTRACT

A region of a imaging subject (20) to be imaged is longer along a translation axis (36) than an imaging field of view (40). The imaging subject (20) and a radio frequency coil (30) are translated together along the translation axis (36) in an inward direction respective to the scanner (10). The inward translating of the radio frequency coil is stopped at a loaded position (ziS0). Subsequent to the stopping, the imaging subject is further translated in the inward direction while the radio frequency coil remains stationary so that the region of the subject to be imaged translates through a stationary field of view (40) of the stationary radio frequency coil. During the further translating, the region is imaged using the stationary radio frequency coil and the magnetic resonance imaging scanner.

The following relates to the medical imaging arts. It finds particular application in magnetic resonance imaging (MRI), and will be described with particular reference thereto. However, it also finds application in magnetic resonance spectroscopy and other modalities which employ magnetic resonance.

In “whole-body” and some other applications of magnetic resonance imaging, a region of interest of a patient is imaged that is larger than the imaging field of view. For example, in spinal imaging the entire length of the spine from the neck to the tailbone or beyond is imaged; however, the imaging field of view typically is not large enough to encompass this entire spinal region of interest. Accordingly, the patient is moved axially (that is, parallel with the spine) through a plurality of stations. At each station, the axial motion of the patient is stopped, and an image is acquired. If neighboring stations are separated by a distance less than the axial length of the imaging field of view, the images at neighboring stations overlap, enabling an image of the entire spine to be reconstructed. This approach is sometimes called the “multi-station” imaging approach. In another approach, the patient is continuously moved in the axial direction, and imaging is performed during the continuous motion. The resulting images typically contain motion artifacts due to the continuous motion of the patient during the imaging; however, these motion artifacts can be suppressed by suitable data corrections.

In either the multi-station or the continuous motion approach, a problem arises with respect to the radio frequency coil. It is desirable to have the radio frequency coil positioned close to the spine to provide good coil sensitivity to magnetic resonance signals emanating from the spinal region. Typically, a spine coil moves along with the patient during the spinal imaging.

However, this approach has disadvantages. Since the spine coil moves with the patient, it should be long enough to span the entire spinal region to be imaged. Since this length is greater than the imaging field of view, a substantial portion of the spine coil is unused at any given point in the multi-station or continuous motion imaging. The spine coil typically consists of a two-dimensional array of surface coil loops; hence, the extended length results in additional coil loops and associated electrical circuitry. The loops and circuitry that are out of the field of view can interfere with magnetic resonance signals in the field of view, and in some instances can receive and contribute stray signals and noise to the image data. Moreover, placement of this extended spine coil on top of the patient can cause physical discomfort. Placement of the coil on top of the patient can contribute to the feeling of claustrophobia experienced by some imaging patients. Patient movement can also disturb the positioning of a coil laid atop the patient.

Other existing approaches also have disadvantages. For example, a permanently mounted spine coil disposed in the scanner bore occupies valuable bore space, and is difficult to position close to the spinal region. Some magnetic resonance imaging scanners include a cylindrical whole-body coil arranged concentrically with the bore. However, the whole-body coil is not as close to the spinal region as a dedicated local coil, and may provide unsatisfactory imaging quality in spinal imaging. Coils that are permanently mounted in the bore are also more difficult to repair.

The present invention contemplates improved apparatuses and methods that overcome the aforementioned limitations and others.

According to one aspect, an apparatus is disclosed, which is operable in conjunction with an associated magnetic resonance scanner for performing imaging or spectroscopy over a region of an associated subject, which region is longer along a translation axis than a field of view. A support is arranged to translate the associated subject along the translation axis into and out of the associated magnetic resonance scanner. A radio frequency coil is coupled with the support to translate along with the support in an inward direction respective to the associated scanner over a loading distance terminating with the coil at a loaded position. The radio frequency coil is held stationary at the loaded position such that further inward translation of the support beyond the loaded position causes translation along the translation axis of the associated subject respective to the stationary radio frequency coil.

According to another aspect, a magnetic resonance imaging system is disclosed, including a magnetic resonance imaging scanner and an apparatus as set forth in the preceding paragraph which is operatively coupled with the scanner to move an extended region of an associated imaging subject through the scanner and relative to the radio frequency coil during an imaging process.

According to another aspect, a method is disclosed for imaging or spectroscopically analyzing a region of an associated subject. The region is longer along a translation axis than a field of view. The associated subject and a radio frequency coil are translated together along the translation axis in an inward direction respective to a magnetic resonance scanner. The inward translating of the radio frequency coil is stopped at a loaded position. Subsequent to the stopping, the associated subject is further translated in the inward direction while the radio frequency coil remains stopped so that the region translates across the stopped radio frequency coil. During the further translating, the region is imaged or spectroscopically analyzed using the stopped radio frequency coil and the magnetic resonance scanner.

According to another aspect, an apparatus is disclosed, which is operable in conjunction with an associated magnetic resonance imaging scanner for performing imaging of an associated imaging subject over a region of the subject that is longer than an imaging field of view. A support means is provided for translating the associated imaging subject into and out of the associated magnetic resonance imaging scanner. A radio frequency coil is disposed with the support means. A means is provided for selectively moving the coil with the support means and an associated subject to a loaded position in the associated scanner and holding the coil stationary at the loaded position as the support means moves the subject relative to the coil.

One advantage resides in reduced spine coil cost and complexity.

Another advantage resides in providing a spine coil arranged close to the spine.

Another advantage resides in improved bore openness and reduced patient claustrophobia.

Another advantage resides in providing a spine coil that is easily accessed and removed.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

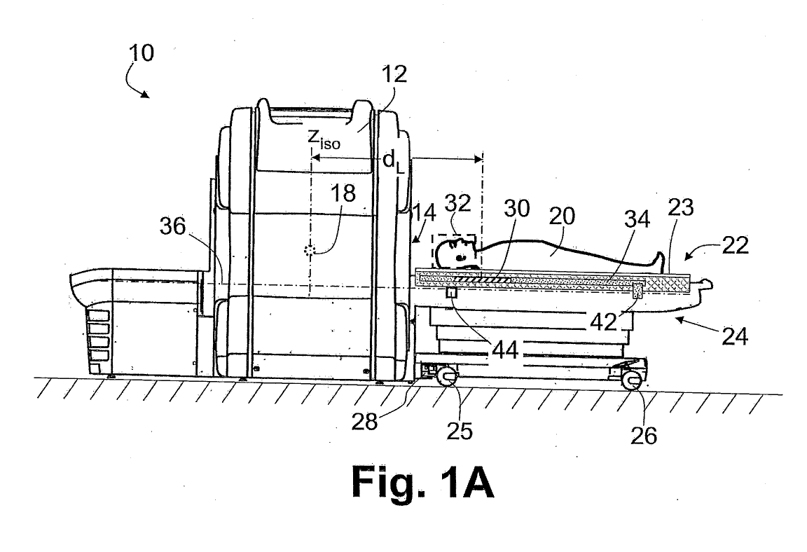

FIGS. 1A, 1B, and 1C diagrammatically show a magnetic resonance imaging scanner including a spine coil disposed with the patient support. FIG. 1A shows the positions of the support and the spine coil just before loading the patient into the scanner. FIG. 1B shows the positions of the support and the spine coil after a loading operation, and just before commencement of spinal imaging. FIG. 1C shows the positions of the support and the spine coil at the end of the spinal imaging. In FIGS. 1B and 1C, a portion of the scanner housing is cut away (as indicated by dashed cutaway lines) to reveal selected components and the imaging subject disposed inside the scanner bore.

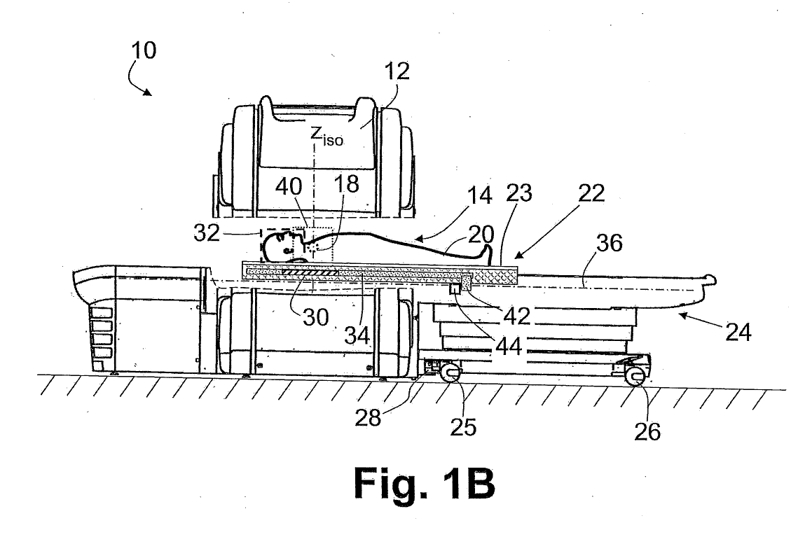

FIG. 2 shows a perspective view of the support.

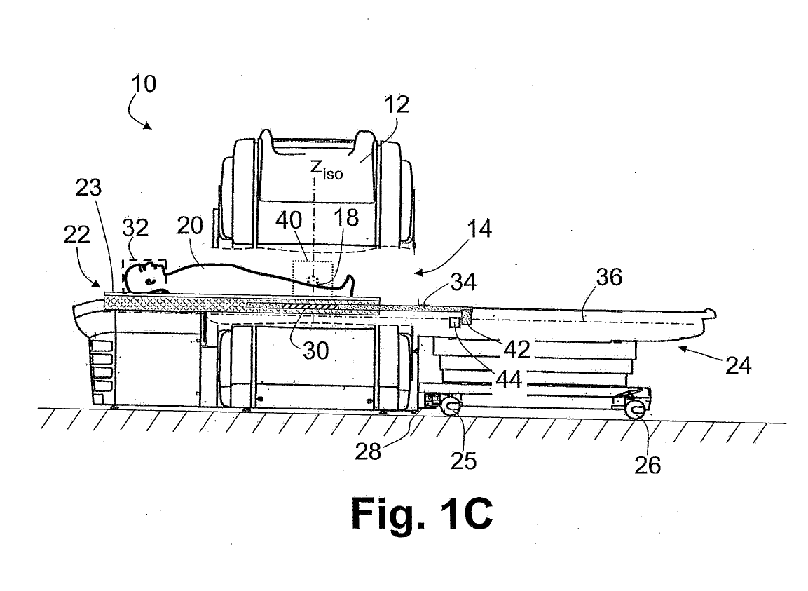

FIG. 3 shows a perspective view of the support with the topmost thin sheet removed, exposing the spine coil.

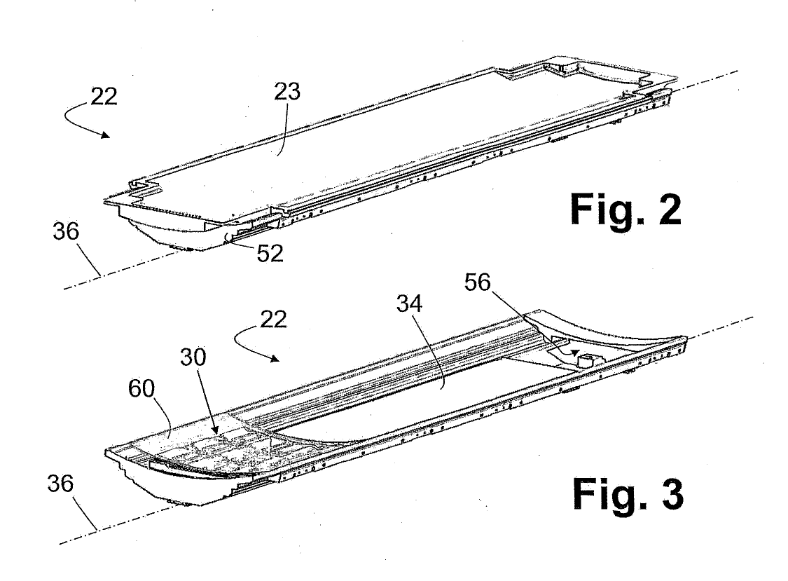

FIG. 4 shows a perspective view of the carrier component of the support.

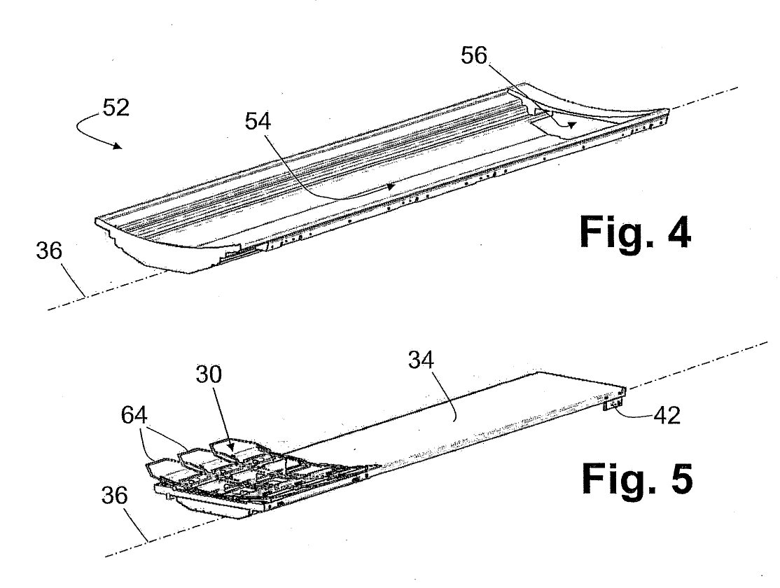

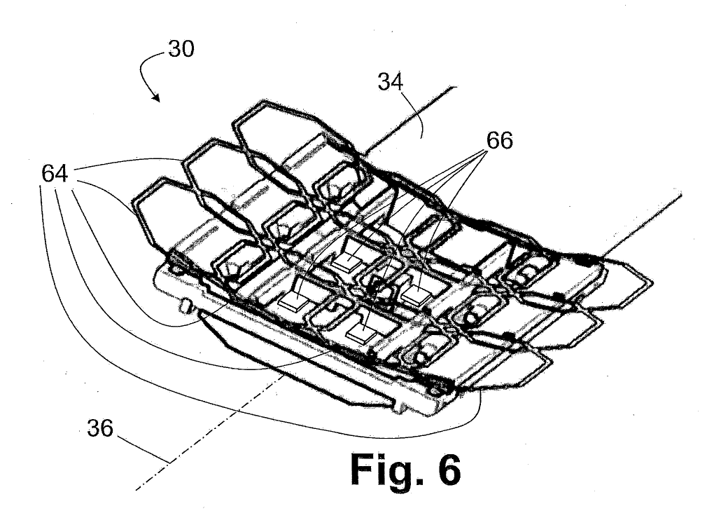

FIGS. 5 and 6 show perspective views of the spine coil and associated mechanical components.

With reference to FIGS. 1A, 1B, and 1C, a magnetic resonance imaging scanner 10 includes a scanner housing 12 that encloses components including at least a main magnet and magnetic field gradient coils. The main magnet is preferably superconducting and cryoshrouded. The scanner housing 12 defines a scanner bore 14 inside which a subject is positioned for imaging. The magnetic field gradient coils are enclosed in the housing 12 or are arranged in the bore 14. The main magnet and the magnetic field gradient coils are configured to provide imaging of suitable quality over an imaging region centered at an isocenter 18 of the scanner 10. In FIGS. 1A, 1B, and 1C, the isocenter 18 is denoted by a dotted circle.

A patient 20 or other imaging subject is disposed on a support 22 that includes a removable thin sheet or tabletop 23. The support 22 is in turn disposed on a trolley 24. In the illustrated embodiment, the trolley 24 is movable on wheels, rollers 25, 26 or so forth, and the trolley 24 is selectably docked with the scanner 10 by a docking mechanism 28. In other embodiments, the trolley 24 is replaced by a stationary couch that is permanently connected with the scanner 10. The trolley 24 is illustrated in the docked position in FIGS. 1A, 1B, and 1C.

During a transmit phase of the magnetic resonance imaging, a radio frequency coil or coils array transmits one or more radio frequency excitation pulses or pulse packets at a magnetic resonance frequency to excite magnetic resonance in the imaging subject 20. During a receive phase of the magnetic resonance imaging, the same coil or coils array, or a different radio frequency coil or coils array, is used to detect the excited magnetic resonance signal emanating from the imaging subject 20. The magnetic resonance signal is optionally spatially localized by applying magnetic field gradients during the transmit phase. Additionally or alternatively, the magnetic resonance signal is optionally spatially encoded by applying magnetic field gradients during the readout phase (typically providing frequency encoding) or during an interval between the transmit and receive phases (typically providing phase encoding). The skilled artisan can readily construct magnetic resonance pulse sequences for providing Cartesian encoding, radial encoding, spiral encoding, or other k-space trajectories. Moreover, the pulse sequence can include spoilers, inversion pulses, refocusing pulses, and other features.

In the embodiment illustrated in FIGS. 1A, 1B, and 1C, two radio frequency coils are illustrated. A spine coil 30 is disposed with the support 22. An optional head coil 32 (diagrammatically shown in phantom) is disposed over the head of the patient 20. The spine coil 30 is connected with an elongated securing member 34, and both the spine coil 30 and the coil securing member 34 are disposed at least partially inside of a hollow region of the support 22.

FIG. 1A shows the positions of the support 22 and the spine coil 30 just before loading the imaging subject 20 into the scanner 10. In this position, the spine coil 30 and the coil securing member 34 are positioned at a head end of the spine and connected or otherwise mounted to move with the support 22.

With continuing reference to FIG. 1A and with further reference to FIG. 1B, during a loading operation the support 22 is moved from the position shown in FIG. 1A to the loaded position shown in FIG. 1B. The support 22 is translated a loading distance d_{L} (labeled in FIG. 1A) along a translation axis 36 (denoted by a dotted-dashed line) in an inward direction respective to the scanner 10, to position the coil 30 in a preselected relationship with the isocenter 18, for example, centered on a vertical plane through the isocenter 18. During the loading operation, the spine coil 30 and the securing member 34 translate together with the support 22 and the imaging subject 20 in the inward direction along the translation axis 36 across the loading distance d_{L}. At the loaded position shown in FIG. 1B, the spine coil 30 is positioned at a loaded position zᵢₛₒ which preferably provides optimal radio frequency coupling with an imaging field of view 40 of the radio frequency coil 30 that includes the isocenter 18 of the magnetic resonance imaging scanner 10.

At the end of the loading operation, the coil 30 is located at the loaded position zᵢₛₒ as shown in FIG. 1B. At this point, the coil 30 and the coil securing member 34 are released from movement with the support 22. In the embodiment of FIGS. 1A, 1B, and 1C, the coil 30 and the coil securing member 34 are connected with the support 22 by friction, and release is achieved by a stop 42 built into the coil securing member 34 contacting a mating stop 44 built into the trolley 24. Other mechanisms can be used for connecting the coil 30 to, and disconnecting the coil 30 from, the support 22. For example, the mating stop can be built into a bridge of the scanner 10 rather than into the trolley 24. Alternatively or additionally, the spine coil 30 can be connected to and disconnected from the support 22 using clamps, locks, or other mechanisms actively driven by magnetic, hydraulic, pneumatic, or other coupling mechanisms.

The imaging starts from the loaded position depicted in FIG. 1B. The support 22 continues to be translated inwardly so as to translate the imaging subject 20; however, the coil 30 remains stationary at the loaded position. Accordingly, the support 22 and the imaging subject 20 translate along the translation axis 36 relative to the stopped coil 30. In some embodiments, the support 22 is moved through a plurality of stations. At each station, the translation of the support 22 is stopped for an imaging time interval, and an image is acquired over the imaging time interval of that portion of the patient 20 lying within the imaging field of view 40 of the coil 30. By spacing neighboring stations by a distance along the translation axis 36 less than the axial length of the imaging field of view 40, the images at neighboring stations overlap, enabling an image of the entire spine to be reconstructed. This is sometimes called the “multi-station” approach. In other embodiments, the patient is continuously translated along the translation axis 36, and imaging is performed during the continuous motion. The resulting imaging data is suitably corrected for motion artifacts, and a spine image is reconstructed.

During imaging, the spine coil 30 is coupled with the imaging field of view 40 at the magnetic resonance frequency, and can be used for exciting magnetic resonance signals, receiving magnetic resonance signals, or both. In some embodiments, a whole-body coil (not shown) disposed in the scanner housing 12 excites magnetic resonance in that portion of the region of interest within the imaging field of view 40, and the spine coil 30 is used to receive the magnetic resonance signal emanating from that portion of the region of interest within the imaging field of view 40.

After the imaging is complete, the support 22, patient 20, spine coil 30, and coil securing member 34 are positioned generally as shown in FIG. 1C. When imaging a smaller area, the support may be stopped between the positions of FIGS. 1B and 1C. To remove the patient 20 after imaging, the support 22 is translated in an outward direction away from the scanner 10. In other words, the outward translational direction is opposite the inward translational direction. The outward translation moves the support 22 back toward the spine coil 30. At a selected point during the outward translation, such as at the position of FIG. 1B, the spine coil 30 and coil securing member 34 are reconnected with the support 22 so that continued outward translation moves both the support 22 and the coil 30 out of the bore 14, until the initial position depicted in FIG. 1A is again reached.

If the spine imaging is to include the head portion, the optional head coil 32 can be used to image the head. In a suitable approach, a field of view of the head coil 32 at least partially overlaps the imaging field of view 40 of the spine coil 30 at the beginning of the spine scan (depicted in FIG. 1B), and the imaging of the head and neck is performed at the beginning of spine scan. In this way, the head and neck image acquired by the head coil 32 at least partially overlaps the spine image acquired using the spine coil 30, enabling a continuous head/spine composite image to be reconstructed.

With reference to FIGS. 2-6, perspective views of a suitable embodiment of the support 22 and spine coil 30 are shown. FIG. 2 shows the assembled support 22 including the tabletop 23 and a carrier component 52. FIG. 4 shows a perspective view the carrier component 52 by itself. The illustrated tabletop 23 is curved to conform with the general curvature of the imaging subject 20. The tabletop 23 is typically removable for cleaning, replacement, or so forth. By removing the tabletop 23, the spine coil 30 is also accessible for removal, repair, or replacement.

In FIG. 3, the tabletop 23 is removed to reveal the spine coil 30 and the coil securing member 34 disposed in an extended slot 54 of the carrier component 52 of the support 22. The slot 54 is parallel with the translation axis 36 to enable the support 22 to translate relative to the stopped spine coil 30 during the spinal imaging. FIG. 3 shows the positions of the spine coil 30 and coil securing member 34 respective to the support 22 corresponding to the initial position of FIG. 1A. The carrier component 52 of the support 22 includes a distal opening 56 at the end distal from the scanner 10, which allows the coil securing member 34 to partially extend outside of the support 22 as the support 22 translates away from the stopped spine coil 30, such as is diagrammatically shown in FIG. 1C. The extended slot 54 and the opening 56 of the carrier component 52 of the support 22 are optionally also large enough to accommodate radio frequency cabling, radio frequency trapping, switching, combining, or other radio frequency components, digital cabling, power cabling, and so forth.

In the view of FIG. 3, the spine coil is covered by an optional coil cover 60, which in the illustrated embodiment is a translucent coil cover. The optional coil cover 60 blocks contact between the tabletop 23 of the support 22 and the spine coil 30. By locating the stopped coil 30 underneath the translating tabletop 23 during imaging, a substantially constant spatial relationship between the region to be imaged and the spine coil 30 is ensured. Optionally, the spine coil 30 can be spring-loaded up against the tabletop 30 to further ensure constancy of this distance as the tabletop 23 translates along the translation axis 36 during imaging.

FIG. 5 shows the spine coil 30 and the coil securing member 34 by themselves, and FIG. 6 shows a closer perspective view of the spine coil 30 and a portion of the coil securing member 34. In both FIGS. 5 and 6, the optional coil cover 60 is removed to more clearly show the features of the underlying coil 30. Because the patient 20 is translated across the stopped coil 30 during imaging, the spine coil 30 can be shorter along the translation axis 36 than the imaged spinal region. For example, in some embodiments the spine coil 30 has a length along the translation axis 36 comparable with the axial size and length of the field of view of the scanner 10, for example less than or about 0.5 meters. In some embodiments, the spine coil 30 includes an array of coil elements. For example, the illustrated spine coil 30 includes a 3×4 array of partially overlapping coil loops 64.

As shown in FIG. 6, analog-to-digital converters 66 are disposed with the radio frequency coil 30 for digitizing analog signals received by the radio frequency coil 30. The analog-to-digital converters 66 translate with the radio frequency coil 30 along the translation axis 36, and stop translating when the coil 30 reaches the loaded position zᵢₛₒ. The analog-to-digital converters 66 are optionally multi-channel analog-to-digital converters to enable each of the coil loops 64 to be communicated away from the coil 30 independently. In other embodiments, the coil loop signals are ported off the coil 30 in analog radio frequency form, and are digitized elsewhere.

Although described with reference to spinal scans, it will be appreciated that the imaging techniques and the apparatuses described herein are readily applied to imaging over other regions that are longer along the translation axis 36 than the imaging field of view 40. For example, the disclosed techniques and apparatuses are applicable to whole-body scans generally, to scans of the arms or legs, extended torso scans, and so forth.

When the spine coil 30 is not to be used in a spinal or other extended field-of-view imaging procedure, the member 34 can be controlled to move the coil 30 out of the field of view. For example, when imaging a region of the subject, the coil can be locked in the position of FIG. 1A. Moreover, while imaging applications have been described, it is to be understood that the techniques described herein employing the spine coil 30 can be applied to voxel-based magnetic resonance spectroscopy, volume magnetic resonance spectroscopy, and other magnetic resonance applications.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. An apparatus, operable in conjunction with an associated magnetic resonance scanner, for performing imaging or spectroscopy over a region of an associated subject, the region being longer along a translation axis than a field of view, the apparatus comprising:
a support arranged to translate the associated subject along the translation axis into and out of the associated magnetic resonance scanner; and
a radio frequency coil coupled with the support to translate along with the support in an inward direction respective to the associated scanner over a loading distance terminating with the coil at a loaded position, the radio frequency coil being held stationary at the loaded position such that further inward translation of the support beyond the loaded position causes translation along the translation axis of the associated subject respective to the stationary radio frequency coil.

2. The apparatus as set forth in claim 1, wherein the radio frequency coil is disposed at least partially inside of a hollow region of the support.

3. The apparatus as set forth in claim 1, wherein the radio frequency coil is disposed at least partially in or on an extended slot (of the support that is parallel with the translation axis.

4. The apparatus as set forth in claim 1, further including:
at least one analog-to-digital converter disposed with the radio frequency coil for digitizing an analog signal received by the radio frequency coil, the analog-to-digital converter translating with the radio frequency coil.

5. The apparatus as set forth in claim 1, further including:
a head coil disposed over a head of the imaging subject, a field of view of the head coil at least partially overlapping the field of view of the radio frequency coil when the coil is at the loaded position.

6. The apparatus as set forth in claim 1, wherein the radio frequency coil includes:
an array of coil elements.

7. The apparatus as set forth in claim 1, wherein the radio frequency coil has a length along the translation axis of less than or about 0.5 meters.

8. The apparatus as set forth in claim 1, wherein the field of view of the radio frequency coil at the loaded position is centered on an isocenter of the associated scanner.

9. A magnetic resonance imaging system comprising:
a magnetic resonance imaging scanner; and
an apparatus as set forth in claim 1 operatively coupled with the scanner to move an extended region of an associated imaging subject through the scanner and relative to the radio frequency coil during an imaging process.

10. A method for imaging or spectroscopically analyzing a region of an associated subject, the region being longer along a translation axis than a field of view, the method comprising:
translating the subject and a radio frequency coil together along the translation axis in an inward direction respective to a magnetic resonance scanner;
stopping the inward translating of the radio frequency coil at a loaded position;
subsequent to the stopping, further translating the subject in the inward direction while the radio frequency coil remains stopped so that the region translates across the stopped radio frequency coil; and
during the further translating, imaging or spectroscopically analyzing the region using the stopped radio frequency coil and the magnetic resonance scanner.

11. The method as set forth in claim 10, wherein:
the further translating includes translating the subject between a plurality of stations, the translating stopping for an examination time interval at each station; and
the imaging or spectroscopic analysis is performed at each station during the examination time interval.

12. The method as set forth in claim 10, wherein:
the further translating includes continuously translating the subject; and
the imaging or spectroscopic analysis is performed simultaneously with the continuous translating.

13. The method as set forth in claim 10, further including:
subsequent to the imaging or spectroscopic analysis, translating the associated subject in an outward direction respective to the magnetic resonance scanner; and
during the translating in the outward direction, initiating outward translation of the radio frequency coil together with the associated subject

14. The method as set forth in claim 10, wherein the imaging or spectroscopic analysis includes:
acquiring analog magnetic resonance signals using the radio frequency coil;
digitizing the acquired magnetic resonance signals at the coil; and
communicating the digitized magnetic resonance signals away from the coil.

15. The method as set forth in claim 10, wherein the stopping of the inward translating of the radio frequency coil at the loaded position includes:
stopping the radio frequency coil at an isocenter of the magnetic resonance scanner.

16. An apparatus, operable in conjunction with an associated magnetic resonance imaging scanner, for performing imaging of an associated imaging subject over a region of the subject that is longer than an imaging field of view, the apparatus comprising:
a support means for translating the associated imaging subject into and out of the associated magnetic resonance imaging scanner;
a radio frequency coil disposed with the support means; and
a means for selectively moving the coil with the support means and an associated subject to a loaded position in the associated scanner and holding the coil stationary at the loaded position as the support means moves the subject relative to the coil.

17. The apparatus as set forth in claim 16, wherein the radio frequency coil is configured to be connected with the support means during a loading operation that includes translating a support surface in an inward direction respective to the associated magnetic resonance imaging scanner over a loading distance.

18. The apparatus as set forth in claim 17, wherein the radio frequency coil is configured to be disconnected from the support means at the completion of the loading operation so as to allow the support surface to continue translating in the inward direction relative to both the associated scanner and the radio frequency coil.

19. The apparatus as set forth in claim 18, wherein at the loaded position a the imaging field of view of the radio frequency coil includes an isocenter of the associated magnetic resonance imaging scanner.

20. The apparatus as set forth in claim 18, wherein the radio frequency coil is configured to be re-connected with the support means during an unloading operation in which the support translates in an outward direction respective to the associated magnetic resonance imaging scanner, the re-connected coil translating with the support in the outward direction to unload both the support and the coil from the associated scanner.
